(19) United States
(12) Patent Application Publication
(10) Pub. No.: US 2025/0160934 A1
(43) Pub. Date: May 22, 2025

(54) PUNCTURE DEVICE ENDS, PUNCTURE SYSTEMS AND METHODS FOR CONTROLLING PUNCTURE SYSTEMS

(71) Applicant: WUHAN UNITED IMAGING SURGICAL CO., LTD., Wuhan (CN)

(72) Inventors: Zhuo ZHAO, Wuhan (CN); Weilong HUANG, Wuhan (CN); Zuiqiu DUAN, Shanghai (CN)

(73) Assignee: WUHAN UNITED IMAGING SURGICAL CO., LTD., Wuhan (CN)

(21) Appl. No.: 19/031,237

(22) Filed: Jan. 17, 2025

Related U.S. Application Data
(63) Continuation of application No. PCT/CN2023/109255, filed on Jul. 26, 2023.

(30) Foreign Application Priority Data
Jul. 26, 2022 (CN) 202210887797.6
Jul. 26, 2022 (CN) 202221961376.5

(51) Int. Cl. A61B 18/14; A61B 17/00; A61B 18/00

(52) U.S. Cl. CPC A61B 18/1477; A61B 2017/00212; A61B 2018/00184; A61B 2018/00613; A61B 2018/143

(57) ABSTRACT

Embodiments of the present disclosure provide puncture device ends, puncture systems, and methods for controlling the puncture systems. The puncture device end includes an end effector and a plurality of puncture needle mounting assemblies disposed in a circumferential direction along the end effector. At least one of the plurality of puncture needle mounting assemblies includes a first mounting member disposed at the end effector and a second mounting member disposed at a second end of the end effector. The first mounting member is capable of moving in a first direction relative to the end effector, and the second mounting member is capable of being fixedly provided with respect to the end effector, the first direction being defined by the first mounting member and the second mounting member.

### CROSS-REFERENCE TO RELATED APPLICATIONS

This application is a continuation of International Application No. PCT/CN2023/109255, filed on Jul. 26, 2023, which claims priority to Chinese Application No. 202210887797.6, filed on Jul. 26, 2022, and Chinese Application No. 202221961376.5, filed on Jul. 26, 2022, the entire contents of each of which are hereby incorporated by reference.

### TECHNICAL FIELD

The present disclosure relates to the technical field of medical devices, and in particular, to puncture device ends, puncture systems, and methods for controlling puncture systems.

### BACKGROUND

Irreversible electroporation, as a method for treating tumors, involves placing two or more electrodes-embedded needles around the tumor in a specific pattern to destroy tumor cells through an electric field. Compared to traditional treatments such as surgery, radiotherapy, and chemotherapy, it has advantages like minimal physical and psychological trauma to patients, lower side effects, no complications, and no sequelae. Unlike physical therapies based on thermal ablation principles, such as radiofrequency, microwave, cryotherapy, and focused ultrasound, irreversible electroporation destroys tumor cells based on non-thermal biomedical effects. This avoids the “thermal sink effect” in vascular and lymphatic systems and can ablate tumor tissue near thermally sensitive organs, overcoming the limitations of thermal therapies (e.g., radiofrequency ablation, microwave ablation, cryoablation, etc.).

During the procedure, at least two electrode needles are required to be placed near the tumor to form the electric field to kill the tumor. The technique demands extremely precise needle placement, making it highly challenging to perform manually. Despite proven clinical effectiveness, these technical difficulties limit large-scale adoption and compromise the consistency of clinical outcomes.

Therefore, it is desired to provide puncture device ends, puncture systems, and methods for controlling the puncture device to meet the need for automated needle placement in the puncture procedure.

### SUMMARY

One or more embodiments of the present disclosure provide a puncture device end. The puncture device end may include an end effector and a plurality of puncture needle mounting assemblies disposed in a circumferential direction along the end effector. The at least one of the plurality of puncture needle mounting assemblies may include a first mounting member provided at a first end of the end effector and a second mounting member provided at a second end of the end effector. The first mounting member may be capable of moving in a first direction with respect to the end effector, the second mounting member may be capable of being fixedly provided with respect to the end effector, and the first direction may be defined by the first mounting member and the second mounting member.

One or more embodiments of the present disclosure provide a puncture system including an active system configured to receive one or more user inputs and generate one or more control signals and a passive system including a puncture device end as described in any of the preceding embodiments, and a robotic arm. The passive system may execute one or more operations based on the one or more control signals.

One or more embodiments of the present disclosure provide a method for controlling a puncture system, including: obtaining information for an object to be processed of a puncture procedure; determining one or more puncture parameters of the puncture system based on the information for the object to be processed; and configuring a puncture device end of the puncture system based on the one or more puncture parameters.

One or more embodiments of the present disclosure provide non-transitory computer-readable storage medium storing computer instructions. When a computer reads the computer instructions in the storage medium, the computer may perform the method as described in any of the preceding embodiments.

Beneficial effects that may be achieved by embodiments of the present disclosure include but are not limited to, the automated needle placement of a plurality of puncture needle assemblies may be achieved.

### BRIEF DESCRIPTION OF THE DRAWINGS

The present disclosure is further illustrated in terms of exemplary embodiments. These exemplary embodiments are described in detail with reference to the drawings. These embodiments are non-limiting exemplary embodiments, in which like reference numerals represent similar structures, and wherein:

FIG. 1 is a schematic diagram illustrating an exemplary puncture device end according to some embodiments of the present disclosure;

FIG. 2A and FIG. 2B are schematic diagrams illustrating an exemplary puncture needle guiding structure according to some embodiments of the present disclosure;

FIG. 3 is a schematic diagram illustrating an exemplary first retractable structure according to some embodiments of the present disclosure;

FIG. 4 is a schematic diagram illustrating an exemplary puncture needle assembly according to some embodiments of the present disclosure;

FIG. 5A is a schematic diagram illustrating an exemplary inner needle according to some embodiments of the present disclosure;

FIG. 5B is a schematic diagram illustrating an exemplary outer needle according to some embodiments of the present disclosure;

FIG. 6A is a schematic diagram illustrating an exemplary electromagnetic elastic assembly according to some embodiments of the present disclosure;

FIG. 6B is a schematic diagram illustrating an exemplary screw drive assembly according to some embodiments of the present disclosure;

FIG. 7 is a schematic diagram illustrating an exemplary puncture system according to some embodiments of the present disclosure;

FIG. 8 is a schematic diagram illustrating an exemplary puncture system according to some embodiments of the present disclosure;

FIG. 9 is a flowchart illustrating an exemplary process for controlling a puncture system according to some embodiments of the present disclosure;

FIG. 10 is a flowchart illustrating an exemplary process for controlling a puncture system according to some embodiments of the present disclosure;

FIG. 11 is a flowchart illustrating exemplary actions performed by a puncture device end according to some embodiments of the present disclosure; and

FIG. 12 is a schematic diagram illustrating a relationship between a first position and a second position according to some embodiments of the present disclosure.

### DESCRIPTION OF REFERENCE SIGNS IN THE ACCOMPANYING DRAWINGS

1 represents an end effector; 2 represents a puncture needle mounting assembly; 3 represents a puncture needle assembly; 4 represents a robotic arm; 5 represents a connecting rod; 11 represents a first end of the end effector; 12 represents a second end of the end effector; 14 represents a second mounting hole; 16 represents a first mounting hole; 21 represents a first mounting member; 22 represents a second mounting member; 23 represents a puncture needle mounting slot; 24 represents a puncture needle guiding structure; 25 represents a first retractable structure; 31 represents an inner needle; 32 represents an outer needle; 241 represents a guiding hole; A represents a first direction; 311 represents an inner needle body; 312 represents an inner needle cap; 321 represents an outer needle body; 322 represents an outer needle cap; 3121 represents a second retractable structure; 3221 represents a depression; 610 represents a first housing; 611 represents a holding section; 612 represents a guiding section; 620 represents an electromagnet; 630 represents a reset spring; 640 represents a connecting section; 650 represents a drive assembly; 660 represents a second housing; 670 represents a screw; 680 represents a screw nut: 690 represents a telescoping block; 651 represents a motor; 652 represents a drive belt; 653 represents a drive wheel; 1201 represents a first puncture needle assembly; 1202 represents a second puncture needle assembly; 1203 represents a tumor; 1204 represents a first plane; 1205 represents a second plane; 1206 represents a first position; and 1207 represents a second position.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise”, “comprises”, “comprising”, “include”, “includes”, and/or “including”, merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

During irreversible electroporation procedures, all electrode needles are required to be parallel or nearly parallel (e.g., an angle between two electrode needles is within a range of) 0°˜±5°, a connection line connecting tips of two electrode needles is perpendicular or nearly perpendicular to the electrode needle bodies (e.g., an angle between the connection line connecting tips of the two electrode needles and the electrode needle bodies is within a range of) 0°˜±5°, and a distance between the electrode needles is subject to strict distance limitations. Therefore, the requirements for the placement of the needles are strict.

Manual operation would require repeated confirmation of the image of the object to be operated on (e.g., a tumor) acquired by the scanner throughout the needle placement process and would require a high level of operator experience. As a result, manual needle placement is not only inefficient but also personnel-dependent, which is unconducive to replication.

Based on this, embodiments of the present disclosure provide puncture device ends, puncture systems, and methods for controlling the puncture systems to automatically deploy a plurality of needles.

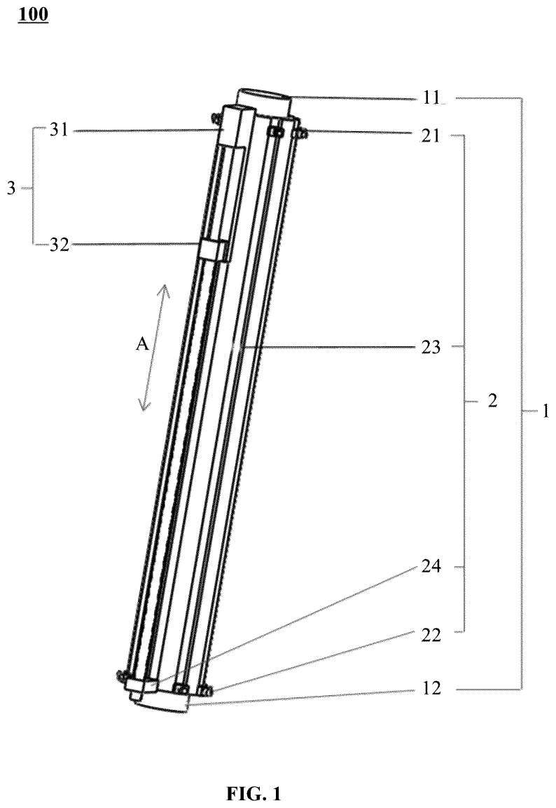

FIG. 1 is a schematic diagram illustrating an exemplary puncture device end according to some embodiments of the present disclosure.

As shown in FIG. 1, in some embodiments, the puncture device end 100 may include an end effector 1 and a plurality of puncture needle mounting assemblies 2.

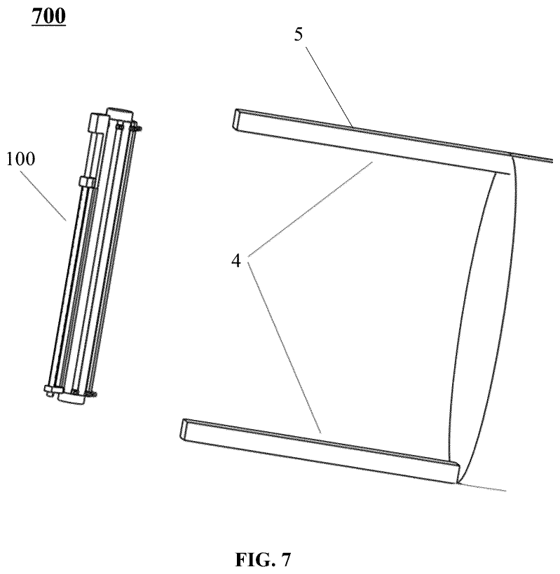

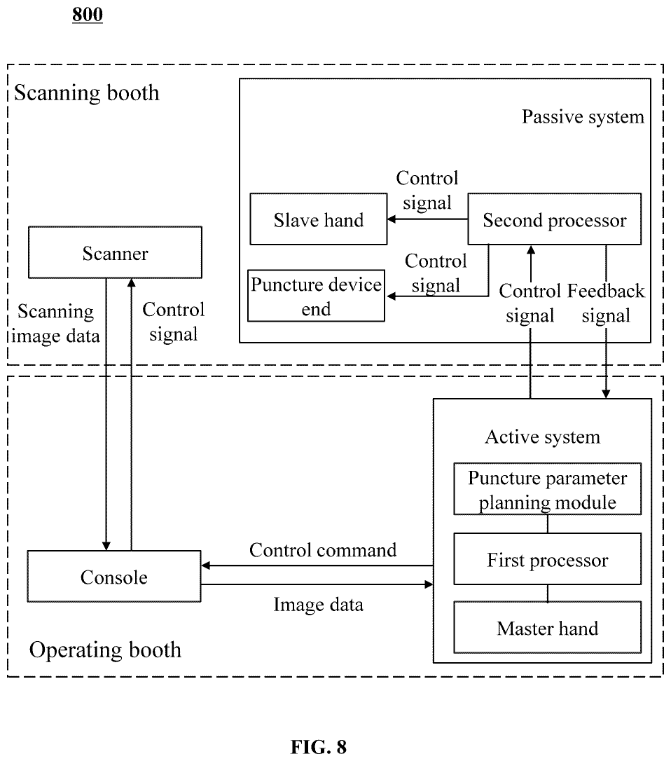

The end effector 1 is configured to mount a plurality of puncture needle mounting assemblies 2 and is configured to be connected to the robotic arm 4. Descriptions regarding the structure of the robotic arm may be found in the description of FIGS. 7 and 8 hereinafter. In some embodiments, the end effector 1 may be cylindrical, or any other shape that can be fitted with a plurality of puncture needle mounting assemblies 2, and all may be included in the scope of the technical conception of the embodiments of the present disclosure. In some embodiments, the end effector 1 is connected with the robotic arm by two ends. In some other embodiments, the end effector 1 may be connected with the robotic arm through one of the two ends.

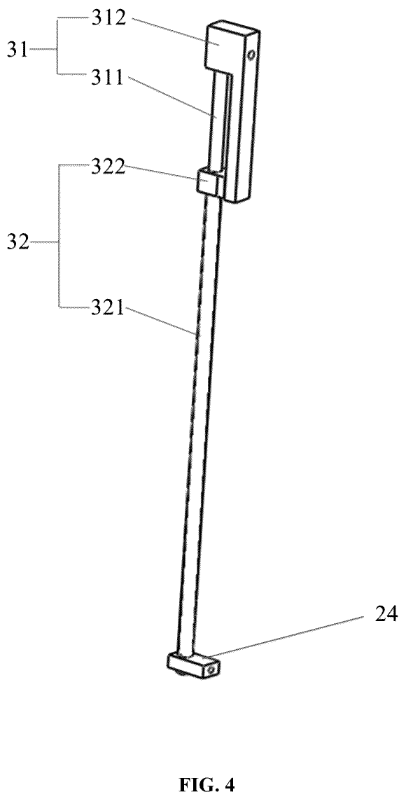

The puncture needle mounting assemblies 2 are configured to mount the puncture needle assembly 3. Descriptions regarding the structure of the puncture needle assembly 3 may be found in the following descriptions of FIGS. 4, 5A, 5B, 6A, and 6B.

In some embodiments, the end effector 1 may be provided with a plurality of puncture needle mounting assemblies 2. It should be understood that the puncture needle assembly 3 may move (forward or backward) in a first direction A relative to the end effector 1 and is also capable of axial movement along the end effector 1. To satisfy a variety of possible circumferential puncture points, a larger number of mounting positions for the puncture needle assembly 3 need to be provided on the end effector 1. In some embodiments, the puncture needle mounting assemblies 2 may be disposed on the end effector 1 along the first direction A. The first direction A refers to a direction parallel to an axis of the end effector 1. More descriptions regarding the first direction A may be found in the related description hereinafter. In some embodiments, the plurality of puncture needle mounting assemblies 2 are disposed in parallel along an axial direction of the end effector 1, to achieve a parallel arrangement of all the puncture needle assemblies 3 and a connection line connecting the tips of the needles is perpendicular to the needle body.

In some embodiments, the puncture device end 100 includes at least 8 puncture needle mounting assemblies 2, and the at least 8 puncture needle mounting assemblies 2 essentially satisfy a variety of possible circumferential puncture points to ensure a smooth puncture operation. It should be noted that the description herein of the puncture device end 100 including at least 8 puncture needle mounting assemblies 2 is provided for exemplary purposes only and is not intended to limit the scope of the present application.

In some embodiments, the plurality of puncture needle mounting assemblies 2 may be disposed along a circumferential direction of the end effector 1. For example, the plurality of puncture needle mounting assemblies 2 are disposed around the circumferential direction of the end effector 1, and their projections are distributed in a variety of shapes along the axial direction of the end effector 1.

In some embodiments, the plurality of puncture needle mounting assemblies 2 are uniformly distributed circumferentially about the axis of the end effector 1, thereby facilitating adjustment of the angular position of the puncture needle assembly 3 by the rotation of the end effector 1. For example, the 8 puncture needle mounting assemblies 2 are evenly spaced at circumferential intervals along the axis of the end effector 1, i.e., one puncture needle mounting assembly 2 is disposed at every 45° along the circumference of the end effector 1.

In some embodiments, at least one of the plurality of puncture needle mounting assemblies 2 may include a first mounting member 21 provided at a first end 11 of the end effector and a second mounting member 22 provided at a second end 12 of the end effector. The first mounting member 21 is capable of moving in the first direction A with respect to the end effector 1, and the second mounting member 22 is capable of being fixedly provided with respect to the end effector 1. The first direction A is defined by the first mounting member 21 and the second mounting member 22. In some embodiments, the plurality of puncture needle mounting assemblies 2 may all be provided according to the first direction A.

The first mounting member 21 is configured to mount the puncture needle assembly 3 and drive the puncture needle assembly 3. In some embodiments, the first mounting member 21 may be provided at the first end of the end effector 11.

In some embodiments, the first mounting member 21 is capable of moving in the first direction A with respect to the end effector 1. In some embodiments, the first direction A is parallel to a longitudinal axis of the end effector 1, which may be determined based on the first mounting member 21 and the second mounting member 22. For example, a straight line is obtained by connecting the center point of the first mounting member 21 and the center point of the second mounting member 22 mounted in correspondence thereto, and the direction along the straight line may be determined as the first direction A. The first end 11 of the end effector refers to one of the end portions of the end effector 1 along the first direction A, and the second end 12 of the end effector refers to the other end portion of the end effector 1 along the first direction A. In some embodiments, the first mounting member 21 may drive the puncture needle assembly 3 during movement.

In some embodiments, the puncture device end 100 may be driven by a pulley drive mechanism, a rope drive mechanism, or a screw drive mechanism to drive the first mounting member 21 to move along the first direction A relative to the end effector 1.

In some embodiments, the first mounting member 21 may move along the first direction A relative to the end effector 1 by the pulley drive mechanism on the end effector 1. In some embodiments, the pulley drive mechanism may include a motor, a master wheel, a follower wheel, and a drive belt, with the master wheel and the follower wheel being provided at opposite ends of the end effector 1, the drive belt being connected to the master wheel and the follower wheel, and the first mounting member 21 being fixedly connected with the drive belt. When the motor drives the master wheel to rotate, the drive belt drives the first mounting member 21 to move along the first direction A relative to the end effector 1. In some embodiments, the first mounting members 21 are fixedly connected with each other to achieve synchronized movement.

In some embodiments, the first mounting member 21 may move along the first direction A relative to the end effector 1 by the rope drive mechanism set on the end effector 1. The rope drive mechanism may include a motor, a master wheel, a follower wheel, and a drive rope. The connection manner is similar to that of the belt drive mechanism and will not be discussed herein.

In some embodiments, the first mounting member 21 may move along the first direction A relative to the end effector 1 by the screw drive mechanism on the end effector 1. In some embodiments, the screw drive mechanism may include a motor, a screw, and a screw nut. The screw nut is mounted at two ends of the end effector 1 and is driven to rotate by the motor, and the screw nut is fixedly connected with the first mounting member 21. When the motor drives the screw to rotate, it may drive the screw nut and the first mounting member 21 to move along the first direction A relative to the end effector 1.

The second mounting member 22 is configured to mount the puncture needle assembly 3 to provide guidance to the movement of the puncture needle assembly 3 to maintain a straight line of movement. In some embodiments, the second mounting member 22 may be provided at the second end of the end effector 12.

In some embodiments, the second mounting member 22 is capable of being fixedly provided relative to the end effector 1. In some embodiments, the second mounting member 22 may direct the movement of the puncture needle assembly 3.

In some embodiments, the movement direction of each puncture needle mounting assembly 2 is parallel to the first direction A, which refers to that an angle between the movement direction of each puncture needle mounting assembly and the first direction A may be within a range of 0°˜±5°.

Some of the embodiments enable movement of the plurality of puncture needle assemblies 3 along the first direction by a plurality of puncture needle mounting assemblies 2, thereby allowing for automated needle placement, which is more stable and reliable than manual needle placement.

In some embodiments, at least one of the plurality of puncture needle mounting assemblies 2 may further include a puncture needle mounting slot 23. In some embodiments, the puncture needle mounting slot 23 is a groove-like structure, which is provided on the end effector 1 along the first direction A. The first mounting member 21 and the second mounting member 22 may be provided inside the groove-like structure, which together with the puncture needle mounting slot 23 form the puncture needle mounting assembly 2.

The puncture needle mounting slot 23 is configured to place the puncture needle assembly 3. In some embodiments, puncture needle mounting slots 23 are distributed equidistant from each other in the circumferential direction of the end effector 1. In some embodiments, the puncture needle mounting slots 23 are parallel to each other. The puncture needle assembly 3, after being provided within the puncture needle mounting slot 23, may be moved in a longitudinal direction along the puncture needle mounting slot 23. In some embodiments, the first direction A may be parallel to the longitudinal direction of the puncture needle mounting slot 23.

In some embodiments, the first mounting member 21 may be provided at a first end of the puncture needle mounting slot 23. The first end of the puncture needle mounting slot 23 corresponds to the first end 11 of the end effector. In some embodiments, the second mounting member 22 may be provided at a second end of the puncture needle mounting slot 23. The second end of the puncture needle mounting slot 23 corresponds to the end effector second end 12.

In some embodiments, a distance between two adjacent first mounting members 21 is equal to a distance between two adjacent second mounting members 22.

Some of the embodiments, by providing the puncture needle mounting slot 23, may provide mounting space for each puncture needle assembly 3, as well as direct the movement of the puncture needle assemblies 3 to stably move along a straight line. In addition, the entire puncture needle end has features of neat arrangement and compact structure.

In some embodiments, a puncture needle guiding structure 24 may be provided on the second mounting member 22. The puncture needle guiding structure 24 is configured to guide the movement of the puncture needle assemblies 3.

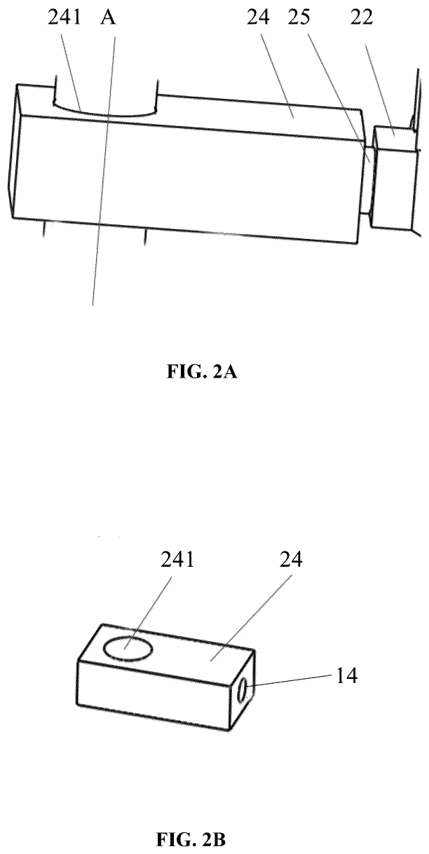

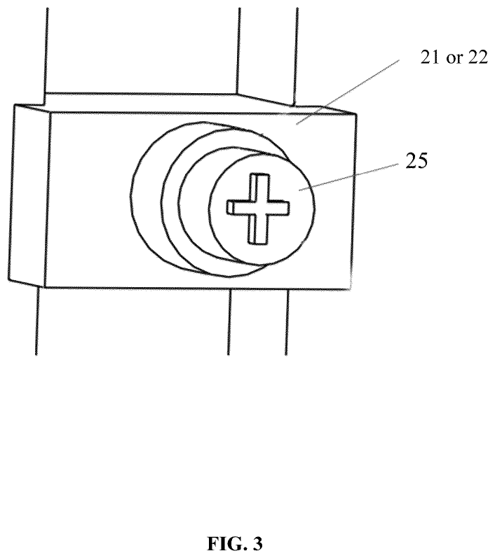

In some embodiments, as shown in FIGS. 2A and 2B, when the puncture needle guiding structure 24 is provided on the second mounting member 22, a guiding hole 241 may be provided in the puncture needle guiding structure 24, and an axial direction of the guiding hole 241 is parallel to the first direction A. The puncture needle assembly 3 may pass through and move in the guiding hole 241. The guiding hole 241 may guide the movement of the puncture needle assemblies 3. In some embodiments, as shown in FIG. 2B, a second mounting hole 14 may be provided in the puncture needle guiding structure 24. The second mounting hole 14 is configured to mount a first retractable structure 25, and the puncture needle guiding structure 24 may be connected with the second mounting member 22 through the second mounting hole 14.

In some embodiments, a C-shaped guiding groove (not shown in the figures) may be provided on the puncture needle guiding structure 24, with the axis direction of the guiding groove being parallel to the first direction A. The puncture needle assemblies 3 may be seated directly from the side portion into the guiding groove and move in the guiding groove. The guiding groove may guide the movement of the puncture needle assemblies 3.

In some embodiments, the first mounting member guide structure may be provided on the end effector 1. For example, the first mounting member guiding structure may include two guide rails parallel to each other and provided on two sides of the end effector 1 corresponding to the puncture needle mounting slot 23, with the first mounting member 21 being provided between the two guide rails. As another example, the first mounting member guiding structure may be a guiding ring and socketed to the end effector 1. The guiding ring is fixedly connected with each first mounting member 21, thereby realizing a relative fixed distance between first mounting members 21. When the guiding ring is moved on the end effector 1, the first mounting members 21 move synchronically.

In some embodiments, the puncture needle guiding structure 24 may be provided on the second mounting member 22, and the first mounting guiding structure may be provided on the end effector 1.

In some embodiments, the puncture needle guiding structure 24 may be provided on the second mounting member 22, and at least one of the plurality of puncture needle mounting assemblies 2 may include the puncture needle mounting slot 23.

In some embodiments, as shown in FIG. 3, the first retractable structure 25 may be provided on the first mounting member 21 and/or the second mounting member 22. FIG. 3 is a schematic diagram illustrating an exemplary first retractable structure according to some embodiments of the present disclosure.

The first retractable structure 25 is configured to adjust a distance between the first mounting member 21 and/or the second mounting member 22 and the end effector 1.

In some embodiments, the first retractable structure 25 may be a multi-section retractable antenna structure. In some embodiments, the first retractable structure 25 may be a hydraulic cylinder, a pneumatic cylinder, or other components that enable retraction.

In some embodiments, one end of the first retractable structure 25 may be fixedly connected with the first mounting member 21, and the other end of the first retractable structure 25 may be fixedly connected with the end effector 1. In some embodiments, one end of the first retractable structure 25 may be fixedly connected with the second mounting member 22, and the other end of the first retractable structure 25 may be detachably connected with the end effector 1.

Some of the embodiments, by providing the first retractable structure 25, may adjust the distance between the first mounting member 21 and/or the second mounting member 22 and the end effector 1, thereby adjusting the needle feed direction of the puncture needle assembly. The plurality of puncture needle mounting assemblies that are retracted and adjusted based on the first retractable structure 25 are parallel.

In some embodiments, as shown in FIG. 1, the puncture device end 100 may further include a puncture needle assembly 3 mounted on the puncture needle mounting assembly 2 through the first mounting member 21 and the second mounting member 22.

In some embodiments, different models of puncture needle assemblies may have different depths of discharge, thereby making them suitable for puncturing into a variety of possible tumor positions for discharge therapy. The depth of discharge refers to the depth that may be achieved when the puncture needle assembly is discharged.

In some embodiments, as shown in FIGS. 1 and 4, the puncture needle assembly 3 may include an inner needle 31 and an outer needle 32, and the outer needle 32 may be nested inside the outer needle 32. FIG. 4 is a schematic diagram illustrating an exemplary puncture needle assembly according to some embodiments of the present disclosure. In some embodiments, the puncture needle assembly 3 may be a solid needle.

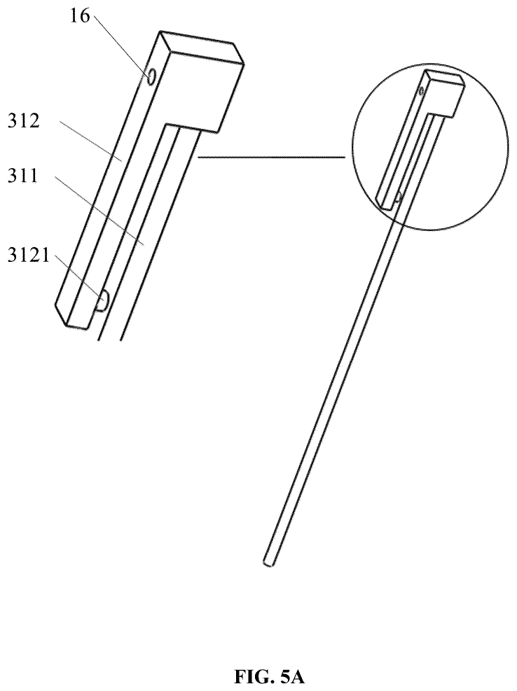

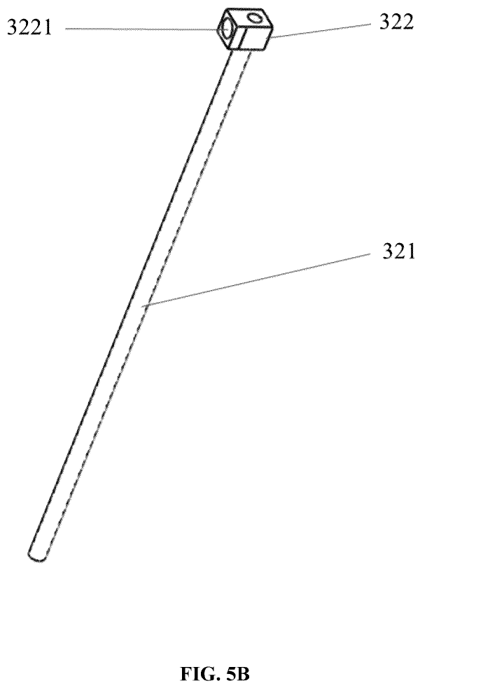

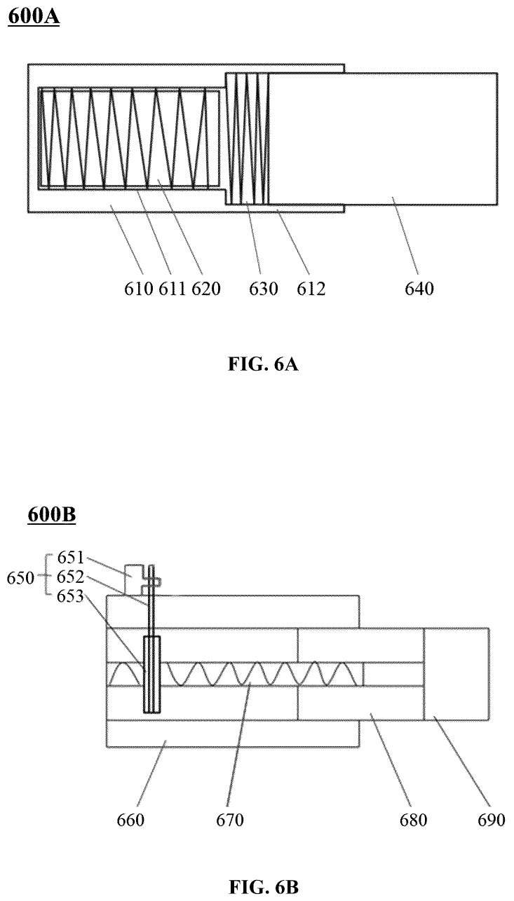

In some embodiments, the outer needle 32 may be selectively connected to or disconnected from the inner needle 31 by a connecting structure. When the outer needle 32 is connected to the inner needle 31, the outer needle 32 and the inner needle 31 may move synchronically. When the outer needle 32 is disconnected from the inner needle 31, the outer needle 32 may be fixed in position and the inner needle 31 may be moved (forward or backward) independently relative to the outer needle 32 in the first direction A. Descriptions regarding the connecting structure may be found in descriptions of FIGS. 5A, 5B, 6A, and 6B hereinafter.

In some embodiments, an end of the inner needle 31 is connected with the first mounting member 21, and the position of the first mounting member 21 is not locked during the discharge of the inner needle 31 so that the inner needle 31 is capable of moving in response to a movement of a puncture object (e.g., there is a rise and fall of the chest during respiration), to avoid the position of the inner needle 31 to deviate (e.g., stabbed deeper or partially withdrawn) during the movement of the puncture object and affecting the surgical effect. At the same time, to avoid the puncture needle assembly being difficult to puncture according to the planned position of the needle placement in the subsequent puncture operation, resulting in difficulty in ensuring that the connection line connecting the needle tips is perpendicular to the needle body and affecting the discharge therapy effect. In some embodiments, the first mounting member 21 may be driven by a pulley drive mechanism, a rope drive mechanism, or a screw drive mechanism to move along the first direction A with respect to the end effector 1, and the first mounting member 21 may be unlocked when the motor controlling either of the pulley drive mechanism, the rope drive mechanism, or the screw drive mechanism releases the clutch thereof. The puncture object refers to a patient who requires the puncture procedure, for example, a patient with a tumor who requires the puncture procedure. The puncture procedure refers to the surgical treatment of inserting a puncture needle into the body of a subject (such as the patient) for treatment. For example, the puncture procedure may include irreversible an electroporation procedure, etc.

In some embodiments, the position of the inner needle 31 is unlocked during the discharge process (e.g., the position of the inner needle 31 may be achieved without locking the first mounting member 21) to allow the inner needle 31 to move in response to the movement of the puncture object.

In some embodiments, as shown in FIGS. 4 and 5A, the inner needle 31 may include an inner needle body 311 and an inner needle cap 312. FIG. 5A is a schematic diagram illustrating an exemplary inner needle according to some embodiments of the present disclosure.

In some embodiments, as shown in FIGS. 4 and 5B, the outer needle 32 may include an outer needle body 321 and an outer needle cap 322. FIG. 5B is a schematic diagram illustrating an exemplary outer needle according to some embodiments of the present disclosure.

In some embodiments, when the outer needle 32 selectively connected to or disconnected from the inner needle 31 by the connecting structure, the connecting structure may be formed by a variety of structures such as a snap connecting structure, a bolt connecting structure, or the like.

In some embodiments, the connecting structure may include a second retractable structure 3121 provided on one of the inner needle cap 312 and the outer needle cap 322, and a depression 3221 provided on the other of the inner needle cap 312 and the outer needle cap 322. The second retractable structure 3121 is configured to cooperate with or separate from the depression 3221. The depression 3221 refers to an inwardly concave structure on the outer needle cap 322, which is configured to cooperate with the second retractable structure 3121 to prevent the inner needle 31 from moving relative to the outer needle 32. The depression 3221 may include, but is not limited to, a slot, a circular hole, a triangular hole, a polygonal hole, or the like.

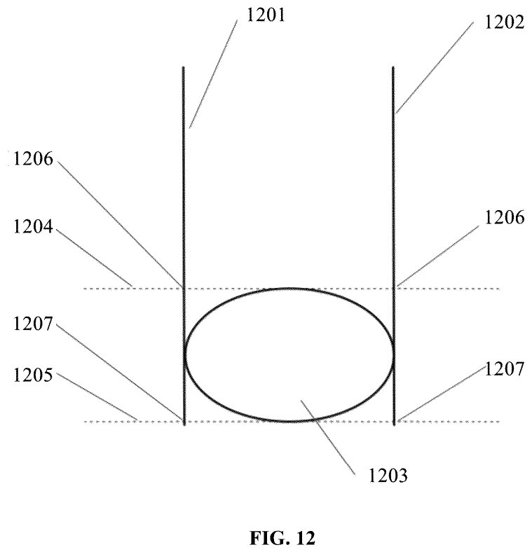

When the puncture is started, the inner needle 31 and the outer needle 32 may be connected by the second retractable structure 3121 so that the inner needle 31, driven by the puncture needle mounting assembly 2, drives the outer needle 32 in the direction of the puncture. After the puncture reaches the first position 1206, the second retractable structure 3121 may be contracted so that the inner needle 31 may move relative to the outer needle 32. At this time, the inner needle 31 may independently continue to downwardly puncture, exposing a specified length of electrode, and discharging between the electrodes to form an electric field. More descriptions regarding the first position 1206 may be found in FIG. 12 and its related description.

Merely by way of example, the second retractable structure 3121 may be lengthened and extended into the depression 3221, which allows for cooperation between the two, thereby allowing the inner needle 31 to move synchronically with the outer needle 32. The second retractable structure 3121 may be shortened and withdrawn from the depression 3221, which allows for separation between the two, thereby allowing the inner needle 31 to move (e.g., forward or backward) independently along the first direction A with respect to the outer needle 32.

In some embodiments, as shown in FIG. 5A, the inner needle cap 312 may include a first mounting hole 16 configured to mount a marker ball (not shown in the figure), which is configured to position the puncture needle assembly 3.

The second retractable structure 3121 may be an electromagnetically elastic component, a screw drive assembly, or other components that may achieve retraction. In some embodiments, the second retractable structure 3121 in the form of the electromagnetically elastic assembly may be as shown in FIG. 6A. FIG. 6A is a schematic diagram illustrating an exemplary electromagnetic clastic assembly according to some embodiments of the present disclosure.

As shown in FIG. 6A, the electromagnetically elastic assembly 600A may include a first housing 610, an electromagnet 620, a reset spring 630, and a connecting section 640. The internal cavity of the first housing 610 is divided into a holding section 611 and a guiding section 612, the electromagnet 620 is placed within the holding section 611, and the reset spring 630 and the connecting section 640 are placed within the guiding section 612. The connecting section 640 may be made of a magnetically conductive material (e.g., iron). In some embodiments, when the electromagnet 620 is energized, the connecting section 640 is attracted toward the electromagnet 620 and compresses the reset spring 630, and the connecting section 640 is withdrawn from the depression 3221, thereby realizing separation between the inner needle 31 and the outer needle 32. After the electromagnet 620 is de-energized, the reset spring 630 returns to its natural length, and if the position of the depression 3221 at this time corresponds to the position of the connecting section 640, the reset spring 630 may push the connecting section 640 into the depression 3221, thereby realizing the connection between the inner needle 31 and the outer needle 32.

Descriptions regarding controlling the electromagnet 620 may be found in FIG. 8 and related descriptions thereof.

In some embodiments, the second retractable structure 3121 in the form of the screw drive assembly may be as shown in FIG. 6B. FIG. 6B is a schematic diagram illustrating an exemplary screw drive assembly according to some embodiments of the present disclosure.

As shown in FIG. 6B, the screw drive assembly 600B may include a drive assembly 650, a second housing 660, a screw 670, a screw nut 680, and a telescoping block 690. In some embodiments, the screw 670 and the screw nut 680 thereon are mounted within the second housing 660, the screw 670 may be driven to rotate by the drive assembly 650, and during rotation of the screw 670, the screw nut 680 may be rotated within the second housing 660 along the screw 670 in an axial direction. When the screw nut 680 moves, it drives the telescoping block 690 connected to the screw nut 680 to move together. In some embodiments, the drive assembly 650 may include a motor 651, a drive belt 652, and a drive wheel 653. The motor 651 may be mounted on the second housing 660, and the motor 651, when rotating at the output end of the motor 651, the drive wheel 653 mounted on the screw 670 is driven to rotate via the drive belt 652, thereby driving the screw to rotate. When the telescoping block 690 is extended into the depression 3221, the inner needle 31 is connected with the outer needle 32, and the inner needle 31 cannot move relative to the outer needle 32. When the telescoping block 690 is withdrawn from the depression 3221, the inner needle 31 is separated from the outer needle 32, and the inner needle 31 may move relative to the outer needle 32.

Some of the foregoing embodiments allow for easy connection or separation between the inner needle 31 and the outer needle 32 through the second retractable structure 3121.

In some embodiments, the outer needle 32 is an electrically insulating needle, the inner needle 31 is an electrode needle, and the outer needle 32 is not electrically conductive when the inner needle 31 is discharged. In some embodiments, the outer needle 32 may include an electrically insulating material, such as insulating steel. In some embodiments, the inner wall of the outer needle 32 is plated with an electrically insulating layer. The inner needle 31 may be electrically energized as a whole, and the second processor may control the depth of discharge by controlling the distance that the inner needle 31 extends from the outer needle 32. More descriptions regarding the second processor may be found below in the present disclosure.

Each puncture device end 100 should include at least two sets of puncture needle assemblies 3 to ensure that at least two inner needles 31 are included therein to enable the formation of an electric field. The inner needles 31 may be arranged in parallel, with a connection line connecting the tips of the needles being perpendicular to the needle body. Two adjacent inner needles 31 may be kept at a certain distance, for example, 1 to 3 cm, from one another.

In some embodiments, the length of discharge of the inner needle body 311 during the operative procedure is controlled by adjusting the length of the inner needle body 311 extending out of the outer needle body 321 so that the electric field formed between the inner needle bodies 311 may be controlled in the region where the tumor is located. For example, the longer the length of the inner needle body 311 extending out of the outer needle body 321, the longer the length of the electric field formed between the inner needle bodies 311 in the first direction A. As another example, the shorter the length of the inner needle body 311 extending out of the outer needle body 321 is, the shorter the length of the electric field formed between the inner needle bodies 311 in the first direction A.

In some embodiments, at least one of the two ends of the end effector 1 is provided with a connecting interface (not shown in the figures).

The connecting interface is configured to realize a mechanical connection and an electrical connection between the end effector 1 and the robotic arm 4. In some embodiments, the first mounting member 21 is electrically connected between the first mounting member 21 and the robotic arm 4 via the connecting interface, to control the first mounting member 21 to move with respect to the end effector 1 along the first direction A. In some embodiments, the first retractable structure 25 is electrically connected to the robotic arm 4 via the connecting interface, thereby adjusting a distance between the first mounting member 21 and/or the second mounting member 22 and the puncture needle assembly 3 by the retraction control of the first retractable structure 25. In some embodiments, the second retractable structure 3121 is electrically connected to the robotic arm 4 via the connecting interface, such that the second retractable structure 3121 is controlled to extend into or out of the depression 3221 to achieve the connection or separation between the inner needle 31 and the outer needle 32.

In some embodiments, the puncture needle assembly 3 is electrically connected to the robotic arm 4 via the connecting interface. In some embodiments, the inner needle 31 is electrically connected to the robotic arm 4 via the connecting interface.

After the puncture device end 100 is mechanically connected and electrically connected with the robotic arm 4 through the connecting interface on the end effector 1, the specific electrical control may be found in the descriptions in FIG. 8 hereinafter.

In some embodiments, the mechanical connection between the end effector 1 and the robotic arm 4 may be a rotatable connection. The robotic arm 4 may include a connecting rod 5 for fixing the end effector 1. The mechanical connection between the connecting rod 5 and the end effector 1 is capable of rotating, thereby facilitating adjustment of the puncture angle of the end effector 1. In some embodiments, the number of connecting rods 5 may be two, facilitating better fixation of the end effector 1. In some embodiments, the connecting rod 5 may be connected with the end effector 1 by a rotary coupling, a rotary connecting disk, or the like, to realize a rotatable connection.

FIG. 7 is a schematic diagram illustrating an exemplary puncture system according to some embodiments of the present disclosure. FIG. 8 is a schematic diagram illustrating an exemplary puncture system according to some embodiments of the present disclosure.

Embodiments of the present disclosure also provide a puncture system 700. In some embodiments, as shown in FIGS. 7 and 8, the puncture system 700 includes an active system and a passive system.

In some embodiments, the active system may be configured to receive one or more user inputs and generate one or more control signals. The user inputs may include operation inputs. The operation inputs refer to user input information related to performing a puncture operation. For example, the operation inputs may include controlling a manipulator to move 10 cm along the first direction A.

In some embodiments, the user inputs may further include planning inputs. The planning inputs refer to puncture parameters of the puncture procedure planned by the user. More descriptions regarding the puncture parameters may be found in related descriptions hereinafter.

In some embodiments, the user may obtain the puncture parameters based on information for an object to be processed of the puncture procedure.

The information for the object to be processed refers to relevant information planned in advance during puncture procedure for the object to be processed (e.g., a tumor). For example, the information for the object to be processed may include the position and shape information of the tumor to be treated during the puncture procedure.

In some embodiments, the information for the object to be processed of the puncture procedure may be obtained in various ways. For example, the puncture parameter planning module may obtain the information for the object to be processed of the puncture procedure by obtaining input information from the user (such as the doctor, etc.). As another example, the puncture parameter planning module may automatically plan and obtain the information for the object to be processed of the puncture procedure based on a medical image. The medical image refers to an image of a human tissue related to the patient's condition, for example, a computed tomography (CT) image of the tumor to be treated.

Merely by way of example, the puncture parameter planning module may obtain the medical image from a memory internal or external to the puncture system 700, process the medical image through a surgical planning model, and obtain the information for the object to be processed of the puncture procedure. The surgical planning model may be a machine learning model, with the medical image as input and the information for the object to be processed as output. The training manner of the surgical planning model may be supervised training, and the training data may be obtained based on historical data.

Understandably, the planning inputs are non-essential in the user inputs, and the puncture parameters may be obtained in other ways. For example, the puncture parameters may be automatically planned and obtained by the puncture system based on the information for the object to be processed during the puncture procedure. The planning inputs may be excluded from the user inputs when the puncture parameters are obtained by automatic planning of the puncture system.

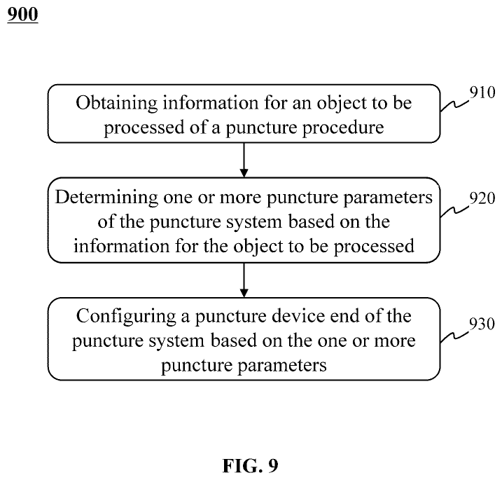

More descriptions regarding automatically planning and obtaining the puncture parameters based on the information for the object to be processed of the puncture procedure may be found in FIG. 9 and related descriptions thereof.

In some embodiments, as shown in FIG. 8, the active system is capable of generating corresponding control signals based on the received user inputs and sending them to the slave system. In some embodiments, the active system may include a puncture parameter planning module, a master hand, and a first processor.

The puncture parameter planning module is configured to determine the puncture parameters of the puncture procedure. For example, the puncture parameter planning module is configured to determine the puncture parameters based on the user inputs. As another example, the puncture system may automatically plan and determine the puncture parameters based on the information for the object to be processed of the puncture procedure.

The master hand is configured to receive a portion of the one or more user inputs. In some embodiments, the master hand may be configured to enable an operator in the operation booth to remotely control a slave hand (i.e., robotic arm 4) in the scanning booth, and guided by the image data from the scanner, the slave hand delivers the puncture device end 100 to the planned target position and performs the operative procedure.

The first processor is configured to process the puncture parameters and the user inputs to generate control signals. In some embodiments, the first processor is configured to communicate with the console and the second processor, respectively, for control of the scanner and the slave system.

In some embodiments, the passive system may include the puncture device end 100 and the robotic arm 4. The passive system is capable of performing operations based on the control signals. For example, the operation performed by the slave system may include moving the robotic arm 4 to the planned target position based on the control signals. For example, the operation performed by the slave system may also include controlling, based on the control signals, the first mounting member 21 of the puncture device end 100 to move along the first direction A, the retraction of the first retractable structure 25, the retraction of the second retractable structure 3121, controlling a puncture component to perform puncture (such as controlling the inner needle 31 to extend the outer needle 32, controlling the inner needle 31 to discharge, etc.), controlling the first mounting member 21 to be locked, or other puncture related operations.

The puncture device end 100 may be a puncture device end 100 as described in any of the preceding embodiments. In some embodiments, the puncture device end 100 is mechanically connected and electrically connected to the robotic arm 4 via connecting interfaces at two ends of the end effector 1. In some other embodiments, the puncture device end 100 realizes the mechanical connection and the electrical connection to the robotic arm through the connecting interface at one of the two ends of the end effector 1.

In some embodiments, the mechanical connection and electrical connection between the slave hand (i.e., the robotic arm 4) and the end 100 of the puncture device is realized through the connecting interface on the end effector 1. The slave hand is provided with a connecting interface matching the connecting interface on the end effector 1, and the end effector 1 may be conveniently connected or separated from the slave hand by plugging.

In some embodiments, the passive system may include a second processor configured to receive the control signals from the first processor and send feedback signals to the first processor. In some embodiments, the second processor may send the control signals to the slave hand and/or the puncture device end 100 to cause the slave hand and/or the puncture device end 100 to complete the puncture needle penetration and the operative procedure.

In some embodiments, the puncture system 700 further includes a scanning system (illustrated in FIG. 8). The scanning system may include a console and a scanner, the scanning system configured to obtain image data based on the control signals.

The scanner is configured to obtain image data, such as an image of a tumor site of a patient. In some embodiments, the scanner is capable of receiving a control signal from a console and acquiring image data based on the control signals, and then transmitting the image data to the console.

The console serves as a control system for the scanner. In some embodiments, the console is capable of receiving control commands from the active system and sending the control signals to the scanner according to the control commands, thereby controlling the scanner to perform a scanning operation to obtain the image data. In some embodiments, the console is capable of receiving image data transmitted by the scanner. In some embodiments, the console may display the received image data. In some embodiments, the console is capable of transmitting the received image data to the active system.

In some embodiments, as shown in FIG. 8, an application scenario 800 may include an operation booth and a scanning booth, with a console and an active system provided in the operation booth, and a scanner and a slave system provided in the scanning booth.

FIG. 9 is a flowchart illustrating an exemplary process for controlling a puncture system according to some embodiments of the present disclosure. In some embodiments, the process 900 may be performed by an active system and/or a slave system in application scenario 800. As shown in FIG. 9, process 900 may include one or more of the following operations.

In 910, information for an object to be processed of a puncture procedure may be obtained. In some embodiments, operation 910 may be performed by a puncture parameter planning module in application scenario 800.

In 920, one or more puncture parameters of the puncture system may be determined based on the information for the object to be processed. In some embodiments, operation 920 may be performed by the puncture parameter planning module in application scenario 800.

The puncture parameters refer to parameters related to the puncturing process of the puncture system during the puncture procedure. In some embodiments, the puncture parameters may include a count of target puncture needle assemblies, positions of the target puncture needle assemblies, etc. In some embodiments, the puncture parameters may include path planning information for various structures related to the puncture procedure. The path planning information may be used to adjust the position of a corresponding structure in the puncture procedure. For example, the path planning information may include, but is not limited to, retracting planning information, movement path planning information, etc. of one or more of the first retractable structure 25, the first mounting member 21, the second mounting member 22, etc., for adjusting the position(s) of target puncture needle assemblies. As another example, the path planning information may also include path planning information for the master hand from the current position to the planned target position, as well as path planning information for the slave hand from the current position to the planned target position. The planned target position refers to a position on the body of the puncture object associated with the tumor. For example, the first position 1206 and the second position 1207 in FIG. 12 may be referred to in the following descriptions of FIG. 12. In some embodiments, the puncture parameters may include electric field planning information. The electric field planning information may include the retracting planning information for the second scalable structure 3121, shape information (e.g., length, width, height) of the electric field, electric field strength and/or electric field duration, etc. In some embodiments, the puncture parameters may also include discharge planning information of the inner needle 31. The discharge planning information may include the discharge current and/or the discharge duration. In some embodiments, the puncture parameters may further include at least one puncture position. The puncture position refers to the position on the body of the subject where the puncture was made (e.g., the entry point).

The target puncture needle assemblies refer to one or more puncture needle assemblies that are required to perform a puncture procedure. In some embodiments, the target puncture needle assemblies may include some or all of the plurality of puncture needle assemblies 3 at the puncture device end.

In some embodiments, the puncture parameter planning module may determine the puncture parameters of the puncture system based on the information for the object to be processed and first historical data. The first historical data may include historical information for the object to be processed and its corresponding historical puncture parameters. For example, the puncture parameter planning module may generate, based on the information for the object to be processed, a first preset table from the first historical data, and determine the puncture parameters of the puncture system by looking up the table.

In some embodiments, the second processor may determine, based on the information for the object to be processed, a count of the target puncture needle assemblies and a target pose for each target puncture needle assembly.

The target pose may include the position of the puncture needle assembly relative to the end effector 1 before the puncture is performed and an inclination angle. It will be appreciated that, since the fundamental purpose of the puncture performed by the puncture needle assembly 3 is to puncture to the tumor position for treatment, the target pose need not overlap completely with the projection of the puncture point along the puncture direction, as long as achieving the purpose of the discharge treatment.

In some embodiments, the second processor may determine, based on the information for the object to be processed, the count of target puncture needle assemblies and the target pose of each target puncture needle assembly in a plurality of ways.

For example, the second processor may determine the count of target puncture needle assemblies based on second historical data, the shape information of the tumor in the information for the object to be processed (e.g., volume, area, etc.) as well as the preset influence range of each puncture point. The second historical data may include historical shape information, a preset influence range of the historical puncture point, and its corresponding historical count of target puncture needle assemblies. Merely by way of example, the puncture parameter planning module may generate, based on the shape information of the tumor in the information for the object to be processed, the preset influence range of each puncture point and the second historical data, a second preset table, and determine the target count of puncture needle assemblies by looking up the table.

As another example, the second processor may determine the puncture position based on tumor position information in the information for the object to be processed, and thus determine the target pose for each target puncture needle assembly based on third historical data. The third historical data may include historical tumor position information and its corresponding historical puncture position. Merely by way of example, the second processor may generate a third preset table based on the tumor position information in the information for the object to be processed and the third historical data, and determine the puncture position by looking up the table. In some embodiments, the second processor may determine the target pose of the target puncture needle assembly after adjusting the determined puncture position. The specific adjustment may be predetermined based on experience, e.g., the adjustment may be to move the puncture position perpendicular to the first direction A toward or away from the end effector 1 by n millimeters and the position after movement may be designated as the target pose, etc.

In some embodiments, the second processor may determine at least one puncture position based on the information for the object to be processed; determine an effective position range for each puncture position; and for each effective position range, determine a target pose of the target puncture needle assembly corresponding to the effective position range based on the effective position range. The specific operation of determining the at least one puncture position based on the information for the object to be processed may be found in the foregoing description related to determining the puncture position based on the tumor position information in the information for the object to be processed.

The effective position range refers to a range of positions surrounding the puncture position where the puncture discharge treatment has the same or similar therapeutic effect as the treatment effect provided by the puncture discharge treatment from the puncture position. For example, the effective position range of a particular puncture position B may be a circular region within 1 cm of the perimeter of that puncture position B.

In some embodiments, the second processor may determine the effective position range of each puncture position based on the puncture needle assembly being in a range of effective discharge treatment. The range of effective discharge treatment refers to a range that may be covered by the electric field formed when the puncture needle assembly undergoes a discharge. The range of the effective discharge treatment may be predetermined based on experience. Merely by way of example, the second processor may have a circular region range centered on the puncture position with a radius of b mm as the effective position range of the puncture position, etc.

In some embodiments, the second processor may determine, based on the effective position range in conjunction with the reachable motion range of the puncture needle assembly, the positions of the plurality of puncture needle assemblies where all of the puncture needle assemblies fall into their respective corresponding effective position ranges with the smallest combined distances as the target pose. The reachable motion range of the puncture needle assembly may be determined based on device-related parameters. The combined distance refers to the distance between each puncture needle assembly and the corresponding puncture position.

Some embodiments of the present disclosure may efficiently and accurately determine the target pose of the target puncture needle assembly by determining the effective position range of each puncture position, thereby determining the target pose of the corresponding target puncture needle assembly, avoiding errors caused by manual positioning.

In 930, a puncture device end of the puncture system is configured based on the puncture parameters. In some embodiments, operation 930 may be performed by the second processor in application scenario 800.

After the puncture parameter planning module determines the puncture parameters, it may send the puncture parameters to the second processor, and the second processor may configure the puncture device end of the puncture system based on the puncture parameters. Specific configurations may include and are not limited to, determining a count of the target puncture needle assembly, a mounting position of the target puncture needle assembly, a position of the end effector 1 that extends out of the target puncture needle assembly after mounting, or the like.

In some embodiments, the second processor may issue instructions to the puncture device end to be assembled based on a determined count of the target puncture needle assemblies and the target pose of each target puncture needle assembly. For each target puncture needle assembly, the second processor may assemble the target puncture needle assembly to the puncture device end, adjust a distance between the first mounting member 21 and/or the second mounting member 22, and the end effector 1 by retractable control of the first retractable structure 25 to adjust the target puncture needle assembly to the corresponding target pose. It can be understood that by adjusting the distance between the first mounting member 21 and/or the second mounting member 22 and the end effector 1 through the first retractable structure 25, the target puncture needle assembly is adjusted relative to the end effector 1. For example, when the distance between the first mounting component 21 and the second mounting component 22, and the end effector 1 is the same, the target puncture needle assembly is parallel to the end effector 1. For example, when the distance between the first mounting member 21 and the second mounting member 22, and the end effector 1 is different, the target puncture needle assembly is inclined relative to the end effector 1. The inclination angle of the target puncture needle assembly relative to the end effector 1 may be determined based on the distance between the first mounting member 21 and the second mounting member 22, and the end effector 1. It is worth noting that the target puncture needle component may be inclined relative to the end effector 1, but the plurality of target puncture needle assemblies need to be parallel to each other to ensure that a stable electric field may be formed at the end of the puncture device, achieving good therapeutic effects.

In some embodiments, the second processor may determine a mounting position of the target puncture needle on the end effector based on the target pose; assemble the target puncture needle assembly to the puncture device end based on the mounting position and move the puncture device end to adjust the target puncture needle assembly to the target pose.

The mounting position refers to the position where the puncture needle assembly 3 is mounted on the end effector 1. In some embodiments, the mounting position may correspond to the puncture needle mounting slot 23 on the end effector 1, i.e., each puncture needle mounting slot 23 is a mounting position. For example, the mounting position may include one of the puncture needle mounting slots 23 in which the puncture needle assembly 3 is mounted on the end effector 1.

The second processor may compare the positions of all of the puncture needle mounting slots 23 on the end effector 1 with the target pose and select a position at which the target puncture needle assembly can reach the target pose after being pushed out as the mounting position.

In some embodiments, for each target puncture needle assembly, the second processor may assemble the target puncture needle assembly to the mounting position on the puncture device end and adjust the target puncture needle assembly to the corresponding target pose.

In some embodiments, the second processor may feed the mounting position back to the user, receive assembly instructions from the user, and then send them to the puncture needle mounting assembly to assemble the target puncture needle assembly to the puncture device end.

In some embodiments of the present disclosure, by determining the mounting position of the target puncture needle assembly on the end effector 1 based on the target pose and assembling the target puncture needle assembly to the puncture device end, the mounting position that matches the target pose may be determined, ensuring that all mounting positions of the target puncture needle assemblies reach the target pose.

In some embodiments, the second processor may adjust the target puncture needle assembly to a corresponding target pose in a plurality of ways.

In some embodiments, the second processor may rotate the end effector 1 to a target angle to cause the target puncture needle assembly provided on the end effector 1 to be adjusted to a corresponding target pose.

The target angle refers to a rotational adjustment angle that enables the puncture needle assembly to reach the target pose.

In some embodiments, the second processor may analyze and process, based on a current rotational control range and the target pose of the target puncture needle assembly on the end effector 1, to determine whether the target pose is located in the current rotational control range. In response, it further performs a rotational action to adjust the angle so that the target puncture needle assembly reaches the target pose, the adjusted angle being the target angle. The rotational control range may be obtained based on the device-related parameters.

In some embodiments, the second processor may also adjust the position of the target puncture needle assembly with respect to the end effector 1 to adjust the target puncture needle assembly to a corresponding target pose. The second processor may analyze and process the target puncture needle assembly on the end effector 1 based on the current retracting control range of the target puncture needle assembly on the end effector 1 and the target pose, determine whether the target pose is located in the current retracting control range. In response, it further performs a retracting action to adjust the retracting length so that the target puncture needle assembly reaches the target pose.

In some embodiments, the second processor may further combine the rotational control range, the retracting control range, and the target pose to determine whether the target pose is located in the combined control range, and in response, further perform the rotational action and the retracting action to adjust the retracting length so that the target puncture needle assembly reaches the target pose. The combined control range refers to a control range of the end effector 1 obtained based on the combination of the rotation control range and the retracting control range.

In some embodiments, the second processor may telescopically control the target puncture needle assembly via the structure of the first mounting 21, the second mounting 22, or the like, adjust the position of the target puncture needle assembly with respect to the end effector 1 to a corresponding target pose. The second processor may determine the adjusted retracting length from fourth historical data. The fourth historical data may include a historical target pose and its corresponding historical retracting length. For example, the second processor may generate a fourth preset table from the fourth historical data and determine the stretch length by looking up the table.

In some embodiments of the present disclosure, by rotating the end effector to the target angle, and/or by adjusting the position of the target puncture needle assembly with respect to the end effector to adjust the target puncture needle assembly to the corresponding target pose, a precise and efficient adjustment may be carried out so that the target puncture needle assembly may accurately reach the target pose and ensure a smooth puncture procedure.

In some embodiments of the present disclosure, by determining the count of the target puncture needle assemblies and the target pose of each target puncture needle assembly based on the information for the object to be processed for assembly and positional adjustments, it is possible to combine the actual condition of the patient with the determination of the exact count and positions of the target puncture needle assemblies to ensure a smooth puncture procedure.

In some embodiments, the manner of controlling the puncture system may further include obtaining one or more control signals and controlling the puncture device end to perform an action based on the control signals. Operations performed at the puncture device end refer to operations related to the puncture procedure, such as a retracting action (e.g., controlling a target puncture needle assembly to retract, etc., via the first mounting member 21 and the second mounting member 22, etc.), a discharging action, a puncture action, etc.

In some embodiments, the second processor may obtain the control signals through the active system, and then control the puncture device end to perform an action based on the control signals. Merely by way of example, when the user clicks the “Discharge” button on the console, the active system may send the control signals for releasing the current to the second processor, automatically controlling the inner needle 31 to act. The active system may send the control signals to release the current to the second processor to automatically control the inner needle 31 to perform the discharge action. When the user manipulates the handle to perform a puncture on the medical image, the active system may also generate the control signals based on the real-time manipulation of the user to control target needles to perform the corresponding puncture action.

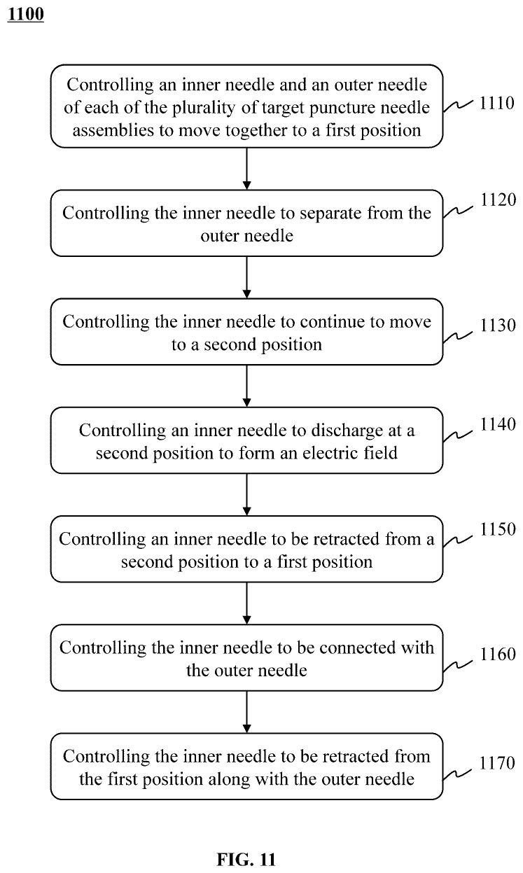

Detailed procedure of controlling the puncture device end to perform the action may be found in descriptions in FIG. 11 hereinafter.

In some embodiments of the present disclosure, by obtaining the information for the object to be processed for the puncture procedure, determining the puncture parameter of the puncture system and configuring the puncture device end, and controlling the puncture device end to perform the puncture action, a plurality of target puncture needle assemblies may be controlled to perform the puncture action synchronically, realizing automated needle placement to meet stringent needle placement requirements, which ensures the parallel arrangement between the target puncture needle assembly and the connection line connecting the needle tips to be perpendicular to the needle body, thus forming a stable electric field to achieve a better therapeutic effect.

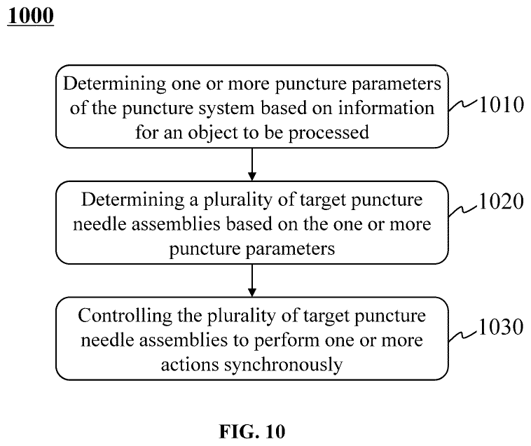

FIG. 10 is a flowchart illustrating an exemplary process for controlling a puncture system according to some embodiments of the present disclosure. As shown in FIG. 10, the process 1000 may include one or more of operation 1010, operation 1020, and operation 1030.

In 1010, one or more puncture parameters are determined based on the information for the object to be processed. In some embodiments, 1010 may be performed by a puncture parameter planning module in application scenario 800.

In 1020, a plurality of target puncture needle assemblies are determined based on the puncture parameters. In some embodiments, operation 1020 may be preformed to determine the count of target puncture needle assemblies and the positions of the target puncture needle assemblies (i.e., which puncture needle assembly or assemblies are specifically selected). In some embodiments, operation 1020 may be performed by the second processor in application scenario 800. In some embodiments, after the puncture parameter planning module determines the puncture parameters, it sends the puncture parameters to the second processor, and the second processor determines that it is necessary to use some or all of the plurality of puncture needle assemblies 3 at the puncture device end as the target puncture needle assembly or assemblies according to the puncture parameters.

In 1030, the plurality of target puncture needle assemblies are controlled to perform one or more actions synchronously. In some embodiments, operation 1030 may be performed by the second processor in application scenario 800. Detailed procedure of operation 930 may be found in descriptions of FIG. 11 hereinafter.

FIG. 11 is a flowchart illustrating exemplary actions performed by a puncture device end according to some embodiments of the present disclosure. In some embodiments, process 1100 may be performed by an active system and/or a slave system in the application scenario 800. As shown in FIG. 11, process 1100 may include one or more of operation 1110, operation 1120, and operation 1130. In some embodiments, process 1100 may further include operation 1140. In some embodiments, process 1100 may further include operation 1150, operation 1160, and/or operation 1170.

In some embodiments, the second processor may control a plurality of target puncture needle assemblies on the puncture device end to perform one or more actions synchronously, which include one or more of the following operations.

In 1110, the inner needle 31 and the outer needle 32 of each of the plurality of target puncture needle assemblies are controlled to move together to a first position 1206. In some embodiments, operation 1110 may be performed by the first mounting member 21. In some embodiments, the movement of the first mounting member 21 is controlled by the second processor.

The first position 1206 refers to a position where the outer needle 32 eventually reaches the edge of the tumor during puncture. At this position, the inner needle 31 may be separated from the outer needle 32, the outer needle 32 stays at the first position 1206, and the inner needle 31 continues to be punctured as shown in FIG. 12. The first position 1206 reached by each outer needles 32 may be in the same plane. For example, as shown in FIG. 12, the first positions 1206 reached by the outer needle 32 of the first puncture needle assembly 1201 and the outer needle 32 of the second puncture needle assembly 1202 are both within the first plane 1204.

In 1110, the inner needle 31 and the outer needle 32 may move synchronically by extending the inner needle 31 and the outer needle 32 of each of the target puncture needle assemblies into the depression 3221 by the second retractable structure 3121.

In some embodiments, the inner needle 31 and the outer needle 32 of each of the target puncture needle assemblies may be synchronized together to enter the puncture object to the first position 1206.

In 1120, the inner needle 31 is controlled to separate from the outer needle 32. In some embodiments, operation 1120 may be performed by the second retractable structure 3121. In some embodiments, the expansion and contraction of the second retractable structure 3121 is controlled by the second processor.

In some embodiments, the inner needle 31 may be separated from the outer needle 32 by the second processor controlling the second retractable structure 3121 to exit the depression 3221. After the inner needle 31 and the outer needle 32 are synchronized for puncturing to reach the first position 1206, the second retractable structure 3121 withdraws from the depression 3221 so that the inner needle 31 is separated from the outer needle 32, the inner needle 31 continues to puncture, and the outer needle 32 may stay at the first position 1206.

In some embodiments, the inner needle 31 and the outer needle 32 of each of the target puncture needle assemblies may be synchronously separated.

In 1130, the inner needle 31 is controlled to continue to move to the second position 1207. In some embodiments, operation 1130 may be performed by the first mounting member 21. In some embodiments, the movement of the first mounting member 21 is controlled by the second processor.

The second position 1207 refers to the planned treatment position that the inner needle 31 ultimately reaches during the puncture. As shown in FIG. 12, after the inner needle 31 has been transported onward to the second position 1207, the nano knife treatment may begin. The second position 1207 reached by each inner needle 31 may be in the same plane. For example, as shown in FIG. 12, the second positions 1207 reached by the inner needle 31 of the first puncture needle assembly 1201 and the inner needle 31 of the second puncture needle assembly 1202 are both within the second plane 1205.

In some embodiments, the inner needle 31 of each of the target puncture needle assemblies may be synchronized to enter the puncture object to the second position 1207.

In some embodiments, a distance between the second position 1207 and the first position 1206 may be determined based on the shape and size of the tumor 1203.

Some of the embodiments may control the outer needles 32 of the plurality of target puncture needle assemblies into the first position 1206 of the puncture object synchronically, and control the inner needles 31 of the plurality of target puncture needle assemblies into the second position 1207 of the puncture object synchronically, thereby realizing automated needle placement of the plurality of target puncture needle assemblies.

In 1140, the inner needle 31 is controlled to discharge at the second position 1207 to form an electric field. In some embodiments, operation 1140 may be controlled by the second processor for the discharge of the inner needle 31.

In some embodiments, the inner needle 31 of each target puncture needle assembly may discharge synchronically.

In some embodiments, movement of the first mounting member 21 relative to the end effector 1 in the first direction A may be driven by a pulley drive mechanism, a rope drive mechanism, or a screw drive mechanism. In some embodiments, the position of the inner needle 31 is unlocked during the discharge process to allow the inner needle 31 to move in response to the movement of the puncture object. In some embodiments, one end of the inner needle 31 is connected to the first mounting member 21, and the first mounting member 21 may move along the first direction during the discharging process of the inner needle 31. When the puncture object performs respiratory movement, the inner needle 31 may be driven to move. The position of the first mounting member 21 on the end effector 1 is unlocked, thus driving the inner needle 31 connected to the first mounting member 21 to move with the respiratory movement of the puncture object, avoiding relative displacement between the puncture needle and tissue caused by the respiratory movement of the puncture object. In some embodiments, the inner needle 31 may be unlocked when the motor controls either the pulley drive mechanism, the rope drive mechanism, or the screw drive mechanism releases the clutch. During the discharging process of the inner needle 31, since the position of each first mounting members 21 is not locked, the inner needle 31 may move synchronically by friction in response to the slight respiration of the puncture object with respect to the end effector 1 in the first direction A, so as to avoid unnecessary damage to the puncture object caused by the inner needle 31 puncturing into a deeper position. For example, the inner needle 31 may puncture deeper into healthy tissue upon further puncturing. In addition, based on the embodiment, it is also possible to avoid that the puncture needle assembly that subsequently performs the puncture action is difficult to puncture according to the planned placement position of the needle and that the connection line connecting the needle tips is difficult to ensure to be perpendicular to the needle body thus affecting the effect of the discharge treatment.

In some of the embodiments, since the outer needle 32 is made of an electrically insulating material, when the outer needle 32 reaches the first position 1206 and the inner needle 31 reaches the second position 1207, an electric field may be formed between the first position 1206 and the second position 1207 through the discharging of the inner needles 31. Therefore, the electric field may be precisely controlled in the region where the tumor 1203 is located, and operative surgery may be carried out on the tumor 1203 to avoid causing damage to the cells outside the electric field. In addition, the outer needle 32 is an electrically insulating needle, and the inner needle 31 is an electrode needle and may be electrically energized. By controlling the distance of the inner needle 31 extending out of the outer needle 32, the discharge depth may be effectively controlled, so that the width of the formed electric field and the discharge range is adjustable, which is more in line with the needs of the puncture surgery.

In 1150, the inner needle 31 is controlled to be retracted from the second position 1207 to the first position 1206. In some embodiments, operation 1150 may be performed by the first mounting member 21. In some embodiments, the movement of the first mounting member 21 is controlled by the second processor.

In some embodiments, the inner needles 31 of the target puncture needle assemblies may be retracted from the second position 1207 synchronically to the first position 1206. In 1160, the inner needle 31 is controlled to be connected with the outer needle 32. In some embodiments, 1160 may be performed by the second retractable structure 3121. In some embodiments, the retracting of the second scalable structure 3121 is controlled by the second processor. In some embodiments, the connection between the inner needle 31 and the outer needle 32 may be accomplished by the second processor controlling the extension into the depression 3221. In some embodiments, after the inner needle 31 has retreated to the first position 1206, the second processor may control the second retractable structure 3121 to extend into the depression 3221 to cause the inner needle 31 to be connected with the outer needle 32.

In some embodiments, the inner needles 31 of the individual target puncture needle assemblies are synchronously connected with the outer needles 32.

In 1170, the inner needle 31 is controlled to be retracted from the first position 1206 along with the outer needle 32. In some embodiments, operation 1170 may be performed by the first mounting member 21. In some embodiments, the movement of the first mounting member 21 is controlled by the second processor.

In some embodiments, the inner needle 31 and the outer needle 32 of each target puncture needle assembly may be retracted together from the first position 1206 synchronically to a position before the puncture is performed.

In some embodiments, the second processor may control the inner needle 31 to be retracted from the outer needle 32 in a plurality of ways. The second processor may control the inner needle 31 and the outer needle 32 to be retracted manually or automatically. For example, after confirming the end of the procedure, the first mounting member 21 and the second mounting member 22 are retracted from the first mounting aperture 16 and the second mounting aperture 14, and the inner needle 31 and the outer needle 32 are released at the same time, then the robotic arm 4 is retracted for the physician manually pulling out the target puncture needle assembly. As another example, after confirming the end of the procedure, the first mounting member 21 guides the inner needle 31 to retract to the first position 1206, and then the second retractable structure 3121 extends to connect the inner needle 31 with the outer needle 32. When the connection between the inner needle 31 and the outer needle 32 is completed, the first mounting member 21 continues to guide the inner needle 31 and the outer needle 32 to retract from the puncture object.

Some of the embodiments may automate the retraction of the inner needle 31 and the outer needle 32 after the operative procedure, reducing dependence on manpower. In addition, since the external needle 32 does not enter the tumor region, the inner needle 31 is retracted through the channel of the outer needle 32 during the retraction of the inner needle 31, which prevents the inner needle 31 from coming in contact with the healthy tissues and prevents the tumor from developing a needle tract metastasis.

In some embodiments, the first retractable structure 25 and the second retractable structure 3121 may be controlled by the second processor to be separated from the target puncture needle assembly, respectively, after the operative procedure, and the target puncture needle assembly may then be manually removed.

One or more embodiments of the present disclosure further provide a non-transitory computer-readable storage medium that stores computer instructions. When a computer reads the computer instructions in the storage medium, the computer performs the method for controlling the puncture system as described in any of the above embodiments of the present disclosure.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment”, “one embodiment”, or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about”, “approximate”, or “substantially”. For example, “about”, “approximate”, or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. A puncture device end, comprising:
an end effector; and
a plurality of puncture needle mounting assemblies disposed in a circumferential direction along the end effector,
wherein at least one of the plurality of puncture needle mounting assemblies includes:
a first mounting member provided at a first end of the end effector; and
a second mounting member provided at a second end of the end effector,
wherein the first mounting member is capable of moving in a first direction with respect to the end effector, the second mounting member is capable of being fixedly provided with respect to the end effector, and the first direction is defined by the first mounting member and the second mounting member.

2. The puncture device end of claim 1, wherein the at least one of the plurality of puncture needle mounting assemblies further includes:
a puncture needle mounting slot configured to place one or more puncture needle assemblies, the first mounting member being provided at a first end of the puncture needle mounting slot, and the second mounting member being provided at a second end of the puncture needle mounting slot.

3. The puncture device end of claim 1, wherein the second mounting member is provided with a puncture needle guiding structure, and/or the end effector is provided with a first mounting member guiding structure.

4. The puncture device end of claim 3, wherein when the puncture needle guiding structure is provided on the second mounting member, a guiding hole is provided on the puncture needle guiding structure, and an axial direction of the guiding hole is parallel to the first direction.

5. The puncture device end of claim 1, wherein a first retractable structure is provided on the first mounting member and/or the second mounting member, and the first retractable structure is configured to adjust a distance between the first mounting member and/or the second mounting member and the end effector, and the plurality of puncture needle mounting assemblies that are retracted and adjusted based on the first retractable structure are parallel.

6. The puncture device end of claim 1, further comprising:
a puncture needle assembly mounted on the plurality of puncture needle mounting assemblies through the first mounting member and the second mounting member,
wherein the puncture needle assembly includes an inner needle and an outer needle, the inner needle is nested inside the outer needle, and the outer needle is selectively connected to or disconnected from the inner needle by a connecting structure.

7. The puncture device end of claim 6, wherein:
the inner needle includes an inner needle body and an inner needle cap;
the outer needle includes an outer needle body and an outer needle cap; and
the connecting structure includes a second retractable structure provided on one of the inner needle cap and the outer needle cap, and a depression provided on the other of the inner needle cap and the outer needle cap, the second retractable structure being configured to cooperate with or separate from the depression.

8. The puncture device end of claim 6, wherein a material of the outer needle includes an electrically insulating material.

9. The puncture device end of claim 6, wherein an end of the inner needle is connected with the first mounting member, and a position of the first mounting member is unlocked during discharge of the inner needle so that the inner needle is capable of moving in response to a movement of a puncture object.

10-11. (canceled)

12. The puncture device end of claim 1, wherein the plurality of puncture needle mounting assemblies are circumferentially and uniformly disposed around an axis of the end effector, and the plurality of puncture needle mounting assemblies are arranged parallel to an axial direction of the end effector.

13. (canceled)

14. A puncture system, comprising:
an active system configured to receive one or more user inputs and generate one or more control signals; and
a passive system including a puncture device end and a robotic arm, wherein the passive system executes one or more operations based on the one or more control signals, and wherein the puncture device end includes:
an end effector; and
a plurality of puncture needle mounting assemblies disposed in a circumferential direction along the end effector,
wherein at least one of the plurality of puncture needle mounting assemblies includes:
a first mounting member provided at a first end of the end effector; and
a second mounting member provided at a second end of the end effector,
wherein the first mounting member is capable of moving in a first direction with respect to the end effector, the second mounting member is capable of being fixedly provided with respect to the end effector, and the first direction is defined by the first mounting member and the second mounting member.

15. The puncture system of claim 14, wherein the active system includes:
a puncture parameter planning module configured to determine one or more puncture parameters of the puncture system;
a master hand configured to receive at least a portion of the one or more user inputs; and
a first processor configured to process the one or more puncture parameters and the one or more user inputs to generate the one or more control signals.

16. (canceled)

17. A method for controlling a puncture system, comprising:
obtaining information for an object to be processed for a puncture procedure;
determining one or more puncture parameters of the puncture system based on the information for the object to be processed; and
configuring a puncture device end of the puncture system based on the one or more puncture parameters.

18. (canceled)

19. The method of claim 17, wherein:
the one or more puncture parameters include a count of target puncture needle assemblies and a target pose of each of the target puncture needle assemblies,
the determining one or more puncture parameters of the puncture system based on the information for the object to be processed includes:
determining the count of the target puncture needle assemblies and the target pose of each of the target puncture needle assemblies based on the information for the object to be processed; and
the configuring a puncture device end of the puncture system based on the one or more puncture parameters includes:
for each of the target puncture needle assemblies, assembling the target puncture needle assembly to the puncture device end and adjusting the target puncture needle assembly to a corresponding target pose.

20. The method of claim 19, wherein the one or more puncture parameters further include at least one puncture position, and the determining the count of the target puncture needle assemblies and the target pose of each of the target puncture needle assemblies based on the information for the object to be processed includes:
determining the at least one puncture position based on the information for the object to be processed;
determining an effective position range for each of the at least one puncture position; and
for each effective position range, determining a target pose of a target puncture needle assembly corresponding to the effective position range based on the effective position range.

21. The method of claim 19, wherein the assembling the target puncture needle assembly to the puncture device end and adjusting the target puncture needle assembly to a corresponding target pose includes:
determining a mounting position of the target puncture needle assembly on an end effector of the puncture device end based on the target pose; and
assembling the target puncture needle assembly to the puncture device end based on the mounting position and moving the puncture device end to adjust the target puncture needle assembly to the target pose.

22. The method of claim 19, wherein the adjusting the target puncture needle assembly to a corresponding target pose includes:
rotating an end effector of the puncture device end to a target angle such that the target puncture needle assembly provided on the end effector is adjusted to the corresponding target pose; and/or
adjusting a position of the target puncture needle assembly with respect to the end effector to adjust the target puncture needle assembly to the corresponding target pose.

23. The method of claim 18, wherein the controlling the puncture device end to perform one or more actions based on the one or more control signals includes:
controlling a plurality of target puncture needle assemblies on the puncture device end to perform the one or more actions synchronously.

24. The method of claim 23, wherein the controlling a plurality of target puncture needle assemblies on the puncture device end to perform the one or more actions synchronously includes:
controlling an inner needle and an outer needle of each of the plurality of target puncture needle assemblies to move together to a first position;
controlling the inner needle to separate from the outer needle; and
controlling the inner needle to continue to move to a second position.

25. (canceled)

26. The method of claim 23, wherein the controlling a plurality of target puncture needle assemblies on the puncture device end to perform the one or more actions synchronously includes:
controlling an inner needle of each of the plurality of target puncture needle assemblies to be retracted from a second position to a first position;
controlling the inner needle to be connected with an outer needle of each of the plurality of target puncture needle assemblies; and
controlling the inner needle to be retracted from the first position along with the outer needle.

27-28. (canceled)
